SEQUENCE LISTING (79 sequences)

SEQ ID NO: 1 - protein, 11 aa, Homo sapiens
QLTPYAQRMER

SEQ ID NO: 2 - protein, 12 aa, Homo sapiens
QKWEAEPVYVQR

SEQ ID NO: 3 - protein, 14 aa, Homo sapiens
ELQQRLEPYADQLR

SEQ ID NO: 4 - protein, 16 aa, Homo sapiens
LHELQEKLSPLGEEMR

SEQ ID NO: 5 - protein, 18 aa, Homo sapiens
QKLHELQEKLSPLGEEMR

SEQ ID NO: 6 - protein, 18 aa, Homo sapiens
LHELQEKLSPLGEEMRDR

SEQ ID NO: 7 - protein, 19 aa, Homo sapiens
DSFHLDEQFTVPVEMMQAR

SEQ ID NO: 8 - protein, 20 aa, Homo sapiens
ARAHVDALRTHLAPYSDELR

SEQ ID NO: 9 - protein, 20 aa, Homo sapiens
LHELQEKLSPLGEEMRDRAR

SEQ ID NO: 10 - protein, 20 aa, Homo sapiens
QKLHELQEKLSPLGEEMRDR

SEQ ID NO: 11 - protein, 22 aa, Homo sapiens
QKLHELQEKLSPLGEEMRDRAR

SEQ ID NO: 12 - protein, 7 aa, Homo sapiens
VLNQELR

SEQ ID NO: 13 - protein, 7 aa, Homo sapiens
YLTWASR

SEQ ID NO: 14 - protein, 8 aa, Homo sapiens
GVTFLLRR

SEQ ID NO: 15 - protein, 9 aa, Homo sapiens
VGYVSGWGR

SEQ ID NO: 16 - protein, 9 aa, Homo sapiens
AFKAWAVAR

SEQ ID NO: 17 - protein, 9 aa, Homo sapiens
LQTDLSEFR

SEQ ID NO: 18 - protein, 10 aa, Homo sapiens
GFSPKDVLVR

SEQ ID NO: 19 - protein, 11 aa, Homo sapiens
DAVSAAFKGLK

SEQ ID NO: 20 - protein, 10 aa, Homo sapiens
WLQGSQELPR

SEQ ID NO: 21 - protein, 10 aa, Homo sapiens
LETPDFQLFK

SEQ ID NO: 22 - protein, 10 aa, Homo sapiens
LGMFNIQHCK

SEQ ID NO: 23 - protein, 11 aa, Homo sapiens
LICQATGFSPR

SEQ ID NO: 24 - protein, 11 aa, Homo sapiens
LRCLAPLEGAR

SEQ ID NO: 25 - protein, 10 aa, Homo sapiens
LGMFNIQHCK

SEQ ID NO: 26 - protein, 9 aa, Homo sapiens
WQEEMELYR

SEQ ID NO: 27 - protein, 11 aa, Homo sapiens
VQPYLDDFQKK

SEQ ID NO: 28 - protein, 11 aa, Homo sapiens
RLGMFNIQHCK

SEQ ID NO: 29 - protein, 14 aa, Homo sapiens
VVAGVANALAHKYH

SEQ ID NO: 30 - protein, 15 aa, Homo sapiens
DSGEGDFLAEGGGVR

SEQ ID NO: 31 - protein, 13 aa, Homo sapiens
VEPLRAELQEGAR

SEQ ID NO: 32 - protein, 12 aa, Homo sapiens
WLQGSQELPREK

SEQ ID NO: 33 - protein, 14 aa, Homo sapiens
VCPFAGILENGAVR

SEQ ID NO: 34 - protein, 16 aa, Homo sapiens
ADSGEGDFLAEGGGVR

SEQ ID NO: 35 - protein, 12 aa, Homo sapiens
ALYLQYTDETFR

SEQ ID NO: 36 - protein, 15 aa, Homo sapiens
LAARLEALKENGGAR

SEQ ID NO: 37 - protein, 15 aa, Homo sapiens
LAARLEALKENGGAR

SEQ ID NO: 38 - protein, 13 aa, Homo sapiens
THLAPYSDELRQR

SEQ ID NO: 39 - protein, 14 aa, Homo sapiens
LLDNWDSVTSTFSK

SEQ ID NO: 40 - protein, 14 aa, Homo sapiens
ITPNLAEFAFSLYR

SEQ ID NO: 41 - protein, 14 aa, Homo sapiens
AGDFLEANYMNLQR

SEQ ID NO: 42 - protein, 16 aa, Homo sapiens
VLGAFSDGLAHLDNLK

SEQ ID NO: 43 - protein, 15 aa, Homo sapiens
QKVEPLRAELQEGAR

SEQ ID NO: 44 - protein, 14 aa, Homo sapiens
YVMLPVADQDQCIR

SEQ ID NO: 45 - protein, 15 aa, Homo sapiens
LSQRFPKAEFAEVSK

SEQ ID NO: 46 - protein, 15 aa, Homo sapiens
YAASSYLSLTPEQWK

SEQ ID NO: 47 - protein, 15 aa, Homo sapiens
YVGGQEHFAHLLILR

SEQ ID NO: 48 - protein, 16 aa, Homo sapiens
SLAELGGHLDQQVEEF

SEQ ID NO: 49 - protein, 17 aa, Homo sapiens
GLPGPPDVPDHAAYHPF

SEQ ID NO: 50 - protein, 15 aa, Homo sapiens
SYFEKSKEQLTPLIK

SEQ ID NO: 51 - protein, 17 aa, Homo sapiens
DSGRDYVSQFEGSALGK

SEQ ID NO: 52 - protein, 17 aa, Homo sapiens
ILGGHLDAKGSFPWQAK

SEQ ID NO: 53 - protein, 16 aa, Homo sapiens
VMPICLPSKDYAEVGR

SEQ ID NO: 54 - protein, 19 aa, Homo sapiens
EVQLVESGGGLVQPGGSLR

SEQ ID NO: 55 - protein, 17 aa, Homo sapiens
VYACEVTHQGLSSPVTK

SEQ ID NO: 56 - protein, 17 aa, Homo sapiens
VTLTCVAPLSGVDFQLR

SEQ ID NO: 57 - protein, 18 aa, Homo sapiens
EIVLTQSPGTLSLSPGER

SEQ ID NO: 58 - protein, 15 aa, Homo sapiens
FNKPFVFLMIEQNTK

SEQ ID NO: 59 - protein, 17 aa, Homo sapiens
SEAEDASLLSFMQGYMK

SEQ ID NO: 60 - protein, 17 aa, Homo sapiens
SEAEDASLLSFMQGYMK

SEQ ID NO: 61 - protein, 17 aa, Homo sapiens
SLHTLFGDKLCTVATLR

SEQ ID NO: 62 - protein, 18 aa, Homo sapiens
TFGSGEADCGLRPLFEKK

SEQ ID NO: 63 - protein, 19 aa, Homo sapiens
VLDAVRGSPAINVAVHVFR

SEQ ID NO: 64 - protein, 18 aa, Homo sapiens
SLEDKTERELLESYIDGR

SEQ ID NO: 65 - protein, 19 aa, Homo sapiens
LYHSEAFTVNFGDTEEAKK

SEQ ID NO: 66 - protein, 19 aa, Homo sapiens
CEGPIPDVTFELLREGETK

SEQ ID NO: 67 - protein, 22 aa, Homo sapiens
TPGAAANLELIFVGPQHAGNYR

SEQ ID NO: 68 - protein, 20 aa, Homo sapiens
QSNNKYAASSYLSLTPEQWK

SEQ ID NO: 69 - protein, 21 aa, Homo sapiens
SEAEDASLLSFMQGYMKHATK

SEQ ID NO: 70 - protein, 20 aa, Homo sapiens
AHVDALRTHLAPYSDELRQR

SEQ ID NO: 71 - protein, 20 aa, Homo sapiens
LTPYADEFKVKIDQTVEELR

SEQ ID NO: 72 - protein, 24 aa, Homo sapiens
GAHGPRPDTVGQRGGSRPSPGPIR

SEQ ID NO: 73 - protein, 29 aa, Homo sapiens
GPPGLPGLKGDPGVPGLPGAKGEVGADGV

SEQ ID NO: 74 - protein, 25 aa, Homo sapiens
AVPPNNSNAAEDDLPTVELQGVVPR

SEQ ID NO: 75 - protein, 25 aa, Homo sapiens
TVRTPGAAANLELIFVGPQHAGNYR

SEQ ID NO: 76 - protein, 23 aa, Homo sapiens
DEPPQSPWDRVKDLATVYVDVLK

SEQ ID NO: 77 - protein, 23 aa, Homo sapiens
QSNNKYAASSYLSLTPEQWKSHR

SEQ ID NO: 78 - protein, 24 aa, Homo sapiens
YRSWVPHTFESELSDPVELLVAES

SEQ ID NO: 79 - protein, 28 aa, Homo sapiens
SASLHLPKLSITGTYDLKSVLGQLGITK

(19) United States
(12) Patent Application Publication
(10) Pub. No.: US 2018/0017555 A1
(43) Pub. Date: Jan. 18, 2018

(54) METHOD FOR DIAGNOSING ARTHROSIS

(71) Applicants: SERVIZO GALEGO DE SAUDE (SERGAS), Santiago de Compostela (A Coruna) (ES); UNIVERSIDADE DE VIGO, Vigo (Pontevedra) (ES); FUNDACION PROFESOR NOVOA SANTOS, A Coruna (ES)

(72) Inventors: Francisco J. BLANCO GARCÍA, Santiago de Compostela (A Coruna) (ES); Cristina RUIZ ROMERO, Santiago de Compostela (A Coruna) (ES); Carolina FERNANDEZ COSTA, A Coruna (ES); Jose Luis CAPELO MARTINEZ, Vigo (Pontevedra) (ES); Florentino FERNANDEZ RIVEROLA, Vigo (Pontevedra) (ES); Miguel REBOIRO JATO, Vigo (Pontevedra) (ES)

(21) Appl. No.: 15/540,249

(22) PCT Filed: Oct. 1, 2015
(86) PCT No.: PCT/ES2015/070717

§ 371 Date: Sep. 18, 2017

(30) Foreign Application Priority Data
Oct. 1, 2014 (ES) P 201431445

(51) Int. Cl. G01N 33/564; G01N 33/68

(52) U.S. Cl. CPC G01N 33/564; G01N 33/6848; G01N 2800/102

(57) ABSTRACT

The present invention relates to a peptide pattern characteristic of subjects who suffer from arthrosis and the use of said peptide pattern in the diagnosis of said disease. Therefore, the present invention also relates to an in vitro diagnostic method and a kit for implementing said method.

The present invention relates to a peptide pattern characteristic of subjects who suffer from arthrosis and the use of said pattern in the diagnosis of said disease. Therefore, the present invention also relates to an in vitro diagnostic method and a kit for implementing said diagnostic method. Therefore, the invention belongs to the technical field of disease diagnosis, in particular, the diagnosis of rheumatic diseases.

### STATE OF THE ART

Arthrosis is a rheumatic pathology that damages articular cartilage. Joints are the skeletal components that allow movement and, consequently, functional autonomy, and are formed by the junction of two bones through the articular capsule. In general, there is a fluid called synovial fluid inside them, which is produced by the synovial membrane. The ends of the bones that come together to form the joint are coated with articular cartilage.

When this articular cartilage is injured, it causes pain, rigidity and functional impairment. Normally, arthrosis is located in the cervical and lumbar spine, and in some joints of the shoulder and the fingers, the hip, the knees and the joint of the head of the big toe.

This rheumatic disease is not hereditary, as there is no fixed hereditary pattern as in the case of haemophilia, for example, but it does have a genetic risk component that, together with other factors, may cause it to appear more frequently in subjects with a family history thereof. In Spain, arthrosis affects 10% of the general population, and represents almost one-fourth of all patients who visit rheumatologists.

Currently, the diagnosis of arthrosis is based on the evaluation of the symptoms and the physical examination of the patients by the physicians. They assess the patients' symptoms, where are they located, what type of pain they have, under which circumstances it improves (e.g., with rest) or gets worse (when going upstairs or downstairs, when opening or closing water taps, etc.), etc. They also ask about other diseases that the patients may have, what treatments are they receiving, and whether or not they or any relatives suffer, or have suffered, from any type of rheumatic disease, trauma or prior articular cartilage injury.

X-rays make it possible to confirm the diagnosis of arthrosis, since they show the radiological changes in the joints typical of arthrotic processes. Radiological studies may be used to determine the severity of arthrosis in a more precise manner.

Blood tests have no utility for diagnosing arthrosis. All the results determined are always normal, including the so-called “rheumatic evidence”, i.e. they do not help to conclude that said subjects suffer from the disease. The only advantage of performing said tests is to confirm the diagnosis of arthrosis when they are normal, since this makes it possible to discard other rheumatic diseases that do produce some alterations in the laboratory tests. For example, in arthritis, the erythrocyte sedimentation rate, the rheumatoid factor and other rheumatic evidences are altered; in gout, uric acid levels are high, etc. Thus far, no diagnostic markers have been identified in the patients' serum or synovial fluid that allows the diagnosis or follow-up of arthrosis.

However, significant progress is being made in the study of the so-called biological markers of arthrosis. These are products released from the articular cartilage into the synovial fluid, the urine or the blood during the synthesis and destruction of the cartilage. Some of them are derivatives of keratan sulfate, chondroitin sulfate, hyaluronic acid or the cartilage oligomeric matrix protein (COMP). Increased knowledge about the biochemical markers of arthrosis would allow for a better diagnosis and earlier intervention in the treatment of the disease, in addition to facilitating the development of drugs, initiating improved therapies, allowing for better medicament options, offering safe dosage options and, finally, reducing health care costs. Consequently, there is a need in the state of the art to identify markers of arthrosis that may allow for an early diagnosis of the disease in a simple, reliable, safe manner.

### DESCRIPTION OF THE INVENTION

The authors of the present invention have discovered that subjects who suffer from arthrosis present a peptide pattern that is not present in healthy subjects, or in subjects who suffer from another type of rheumatic disease, such as psoriatic arthritis (PsA) and rheumatoid arthritis (RA), which makes said peptide pattern a useful tool for the specific diagnosis of arthrosis. In order to obtain the aforementioned pattern, the authors started with serum samples from patients diagnosed with arthrosis and performed a chemical depletion with dithiothreitol (DTT) and acetonitrile (ACN), in order to subsequently perform a tryptic digestion accelerated by means of ultrasound. Thereafter, the resulting peptides were retained in a commercial C18 tip and eluted with different amounts of ACN. Finally, each eluted fraction was analysed in quintuplicate by means of MALDI-TOF/TOF mass spectrometry (Example 1), to obtain the masses of the peptides characteristic of the peptide pattern present in subjects who suffer from arthrosis. The subsequent validation of the peptide pattern obtained (Example 2) confirmed the utility of this pattern for the diagnosis of arthrosis.

The different inventive aspects developed by the inventors are described below.

### Peptide Pattern of the Invention and Uses Thereof

The authors of the present invention have discovered that patients with arthrosis present a characteristic peptide pattern that is not present neither in subjects with rheumatic diseases other than arthrosis (such as psoriatic arthritis or rheumatoid arthritis), nor in healthy subjects.

Therefore, one aspect of the invention relates to a peptide pattern, hereinafter peptide pattern of the invention, that comprises the following peptides: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11.

In the present invention, “peptide pattern” is understood to mean the set or group of peptides present in a biological sample from a subject that is characteristic of his/her physiological state. Within the context of the present invention, the peptide pattern described is characteristic of subjects who suffer from arthrosis.

In the present invention, “peptide” is understood to mean the molecule formed by the binding of several amino acids through peptide bonds which have, approximately, between 2 and 100 amino acids. As a guideline, if the peptide comprises between approximately 2 and 15 amino acids, it is called oligopeptide, and, if it comprises between approximately 16 and 100 amino acids, it is called polypeptide.

As shown in the examples included in the present description, the peptide pattern of the invention was obtained by means of MALDI-TOF/TOF mass spectrometry, reason, in addition to being characterized by their specific amino acid sequence, the peptides of the invention are also characterized by their mass/charge value and their molecular weight [see Table 1], which are standard parameters in this type of technique.

TABLE 1
Mass/charge ratio values and molecular weights
obtained by means of mass spectrometry for each
peptide comprised within the peptide pattern
of the invention.

| | Mass/ | Molecular | SEQ | |
|---|---|---|---|---|
| | charge | weight | ID | Peptide |
| | (m/z) | (Da) | NO: | sequence |
| Peptide 01 | 1392.7526 | 1391.7526 | 1 | QLTPYAQRMER |
| Peptide 02 | 1532.8060 | 1531.806 | 2 | QKWEAEPVYVQR |
| Peptide 03 | 1758.9790 | 1757.979 | 3 | ELQQRLEPYADQLR |
| Peptide 04 | 1910.0176 | 1909.0176 | 4 | LHELQEKLSPLGEEM |
| | | | | R |
| Peptide 05 | 2165.1272 | 2164.1272 | 5 | QKLHELQEKLSPLGE |
| | | | | EMR |
| Peptide 06 | 2180.1445 | 2179.1445 | 6 | LHELQEKLSPLGEEM |
| | | | | RDR |
| Peptide 07 | 2280.1501 | 2279.1501 | 7 | DSFHLDEQFTVPVEM |
| | | | | MQAR |
| Peptide 08 | 2291.2260 | 2290.226 | 8 | ARAHVDALRTHLAPY |
| | | | | SDELR |
| Peptide 09 | 2407.2880 | 2406.288 | 9 | LHELQEKLSPLGEEM |
| | | | | RDRAR |
| Peptide 10 | 2436.2922 | 2435.2922 | 10 | QKLHELQEKLSPLGE |
| | | | | EMRDR |
| Peptide 11 | 2663.4314 | 2662.4314 | 11 | QKLHELQEKLSPLGE |
| | | | | EMRDRAR |

Table 1 also shows the peptide sequence of the peptides that comprise the pattern of the invention.

In a particular embodiment, the peptide pattern of the invention further comprises at least one peptide selected from the group consisting of the peptides shown in Table 2, which are characterized by both their specific amino acid sequence, and their mass/charge value and molecular weight.

TABLE 2
Sequences, mass/charge values and molecular weights obtained by means
of mass spectrometry for each additional peptide that may be used in
combination with the peptide pattern of the invention.

| | Mass/charge | Molecular | SEQ ID | |
|---|---|---|---|---|
| | (m/z) | weight (Da) | NO: | Peptide sequence |
| Peptide 12 | 871.502 | 870.502 | 12 | VLNQELR |
| Peptide 13 | 896.490 | 895.490 | 13 | YLTWASR |
| Peptide 14 | 961.627 | 960.627 | 14 | GVTFLLRR |
| Peptide 15 | 980.529 | 979.529 | 15 | VGYVSGWGR |
| Peptide 16 | 1019.604 | 1018.604 | 16 | AFKAWAVAR |
| Peptide 17 | 1108.588 | 1107.588 | 17 | LQTDLSEFR |
| Peptide 18 | 1117.669 | 1116.669 | 18 | GFSPKDVLVR |
| Peptide 19 | 1148.634 | 1147.634 | 19 | DAVSAAFKGLK |
| Peptide 20 | 1213.667 | 1212.667 | 20 | WLQGSQELPR |
| Peptide 21 | 1237.674 | 1236.674 | 21 | LETPDFQLFK |
| Peptide 22 | 1247.652 | 1246.652 | 22 | LGMFNIQHCK |
| Peptide 23 | 1249.655 | 1248.655 | 23 | LICQATGFSPR |
| Peptide 24 | 1255.716 | 1254.716 | 24 | LRCLAPLEGAR |
| Peptide 25 | 1263.683 | 1262.683 | 25 | LGMFNIQHCK |
| Peptide 26 | 1299.609 | 1298.609 | 26 | WQEEMELYR |
| Peptide 27 | 1380.780 | 1379.780 | 27 | VQPYLDDFQKK |
| Peptide 28 | 1403.749 | 1402.749 | 28 | RLGMFNIQHCK |
| Peptide 29 | 1449.800 | 1448.800 | 29 | VVAGVANALAHKYH |
| Peptide 30 | 1465.689 | 1464.689 | 30 | DSGEGDFLAEGGGVR |
| Peptide 31 | 1467.852 | 1466.852 | 31 | VEPLRAELQEGAR |
| Peptide 32 | 1470.813 | 1469.813 | 32 | WLQGSQELPREK |
| Peptide 33 | 1502.810 | 1501.810 | 33 | VCPFAGILENGAVR |
| Peptide 34 | 1518.710 | 1517.710 | 34 | ADSGEGDFLAEGGGVR |
| Peptide 35 | 1519.750 | 1518.750 | 35 | ALYLQYTDETFR |
| Peptide 36 | 1568.914 | 1567.914 | 36 | LAARLEALKENGGAR |
| Peptide 37 | 1569.907 | 1568.907 | 37 | LAARLEALKENGGAR |
| Peptide 38 | 1585.846 | 1584.846 | 38 | THLAPYSDELRQR |
| Peptide 39 | 1612.849 | 1611.849 | 39 | LLDNWDSVTSTFSK |
| Peptide 40 | 1641.892 | 1640.892 | 40 | ITPNLAEFAFSLYR |
| Peptide 41 | 1641.892 | 1640.892 | 41 | AGDFLEANYMNLQR |
| Peptide 42 | 1669.862 | 1668.862 | 42 | VLGAFSDGLAHLDNLK |
| Peptide 43 | 1706.931 | 1705.931 | 43 | QKVEPLRAELQEGAR |
| Peptide 44 | 1707.875 | 1706.875 | 44 | YVMLPVADQDQCIR |
| Peptide 45 | 1736.970 | 1735.970 | 45 | LSQRFPKAEFAEVSK |
| Peptide 46 | 1743.882 | 1742.882 | 46 | YAASSYLSLTPEQWK |
| Peptide 47 | 1745.901 | 1744.901 | 47 | YVGGQEHFAHLLILR |
| Peptide 48 | 1771.885 | 1770.885 | 48 | SLAELGGHLDQQVEEF |
| Peptide 49 | 1786.909 | 1785.909 | 49 | GLPGPPDVPDHAAYHPF |
| Peptide 50 | 1810.992 | 1809.992 | 50 | SYFEKSKEQLTPLIK |
| Peptide 51 | 1815.922 | 1814.922 | 51 | DSGRDYVSQFEGSALGK |
| Peptide 52 | 1825.005 | 1824.005 | 52 | ILGGHLDAKGSFPWQAK |
| Peptide 53 | 1834.946 | 1833.946 | 53 | VMPICLPSKDYAEVGR |
| Peptide 54 | 1864.027 | 1863.027 | 54 | EVQLVESGGGLVQPGGSLR |
| Peptide 55 | 1875.997 | 1874.997 | 55 | VYACEVTHQGLSSPVTK |
| Peptide 56 | 1876.017 | 1875.017 | 56 | VTLTCVAPLSGVDFQLR |
| Peptide 57 | 1884.000 | 1883.000 | 57 | EIVLTQSPGTLSLSPGER |
| Peptide 58 | 1899.060 | 1898.060 | 58 | FNKPFVFLMIEQNTK |
| Peptide 59 | 1906.873 | 1905.873 | 59 | SEAEDASLLSFMQGYMK |
| Peptide 60 | 1922.877 | 1921.877 | 60 | SEAEDASLLSFMQGYMK |
| Peptide 61 | 1932.038 | 1931.038 | 61 | SLHTLFGDKLCTVATLR |
| Peptide 62 | 2012.011 | 2011.011 | 62 | TFGSGEADCGLRPLFEKK |
| Peptide 63 | 2020.138 | 2019.138 | 63 | VLDAVRGSPAINVAVHVFR |
| Peptide 64 | 2153.112 | 2152.112 | 64 | SLEDKTERELLESYIDGR |
| Peptide 65 | 2186.092 | 2185.092 | 65 | LYHSEAFTVNFGDTEEAKK |
| Peptide 66 | 2190.132 | 2189.132 | 66 | CEGPIPDVTFELLREGETK |
| Peptide 67 | 2296.219 | 2295.219 | 67 | TPGAAANLELIFVGPQHAGNYR |
| Peptide 68 | 2315.195 | 2314.195 | 68 | QSNNKYAASSYLSLTPEQWK |
| Peptide 69 | 2344.169 | 2343.169 | 69 | SEAEDASLLSFMQGYMKHATK |
| Peptide 70 | 2348.245 | 2347.245 | 70 | AHVDALRTHLAPYSDELRQR |
| Peptide 71 | 2394.298 | 2393.298 | 71 | LTPYADEFKVKIDQTVEELR |
| Peptide 72 | 2409.281 | 2408.281 | 72 | GAHGPRPDTVGQRGGSRPSPGPIR |
| Peptide 73 | 2565.322 | 2564.322 | 73 | GPPGLPGLKGDPGVPGLPGAKGEVGADGV |
| Peptide 74 | 2602.358 | 2601.358 | 74 | AVPPNNSNAAEDDLPTVELQGVVPR |
| Peptide 75 | 2652.448 | 2651.448 | 75 | TVRTPGAAANLELIFVGPQHAGNYR |
| Peptide 76 | 2670.387 | 2669.387 | 76 | DEPPQSPWDRVKDLATVYVDVLK |
| Peptide 77 | 2695.362 | 2694.362 | 77 | QSNNKYAASSYLSLTPEQWKSHR |
| Peptide 78 | 2790.425 | 2789.425 | 78 | YRSWVPHTFESELSDPVELLVAES |
| Peptide 79 | 2940.755 | 2939.755 | 79 | SASLHLPKLSITGTYDLKSVLGQLGITK |

In another, more particular embodiment, the peptide pattern of the invention further comprises at least one peptide selected from the group consisting of the peptides shown in Table 3, which are characterized by both their specific amino acid sequence, and their mass/charge value and molecular weight. The peptides shown in Table 3 may be used in combination with the peptide pattern of the invention in order to differentiate between arthrotic subjects and normal subjects. In another, more preferred embodiment, arthrotic subjects show an increased presence of the peptides shown in Table 3 as compared to the healthy control subjects.

TABLE 3
Sequences, mass/charge values and molecular weights obtained
by means of mass spectrometry for each additional peptide that
may be used in combination with the peptide pattern of the
invention in order to differentiate between arthrotic subjects
and normal subjects.

| | | Molecular | | |
|---|---|---|---|---|
| | Mass/charge | weight | SEQ ID | |
| | (m/z) | (Da) | NO: | Peptide sequence |
| Peptide 13 | 896.490 | 895.490 | 13 | YLTWASR |
| Peptide 14 | 961.627 | 960.627 | 14 | GVTFLLRR |
| Peptide 15 | 980.529 | 979.529 | 15 | VGYVSGWGR |
| Peptide 16 | 1019.604 | 1018.604 | 16 | AFKAWAVAR |
| Peptide 17 | 1108.588 | 1107.588 | 17 | LQTDLSEFR |
| Peptide 18 | 1117.669 | 1116.669 | 18 | GFSPKDVLVR |
| Peptide 19 | 1148.634 | 1147.634 | 19 | DAVSAAFKGLK |
| Peptide 20 | 1213.667 | 1212.667 | 20 | WLQGSQELPR |
| Peptide 21 | 1237.674 | 1236.674 | 21 | LETPDFQLFK |
| Peptide 24 | 1255.716 | 1254.716 | 24 | LRCLAPLEGAR |
| Peptide 28 | 1403.749 | 1402.749 | 28 | RLGMFNIQHCK |
| Peptide 32 | 1470.813 | 1469.813 | 32 | WLQGSQELPREK |
| Peptide 33 | 1502.810 | 1501.810 | 33 | VCPFAGILENGAVR |
| Peptide 36 | 1568.914 | 1567.914 | 36 | LAARLEALKENGGAR |
| Peptide 38 | 1585.846 | 1584.846 | 38 | THLAPYSDELRQR |
| Peptide 40 | 1641.892 | 1640.892 | 40 | ITPNLAEFAFSLYR |
| Peptide 41 | 1641.892 | 1640.892 | 41 | AGDFLEANYMNLQR |
| Peptide 42 | 1669.862 | 1668.862 | 42 | VLGAFSDGLAHLDNLK |
| Peptide 43 | 1706.931 | 1705.931 | 43 | QKVEPLRAELQEGAR |
| Peptide 51 | 1815.922 | 1814.922 | 51 | DSGRDYVSQFEGSALGK |
| Peptide 52 | 1825.005 | 1824.005 | 52 | ILGGHLDAKGSFPWQAK |
| Peptide 53 | 1834.946 | 1833.946 | 53 | VMPICLPSKDYAEVGR |
| Peptide 54 | 1864.027 | 1863.027 | 54 | EVQLVESGGGLVQPGGSLR |
| Peptide 55 | 1875.997 | 1874.997 | 55 | VYACEVTHQGLSSPVTK |
| Peptide 56 | 1876.017 | 1875.017 | 56 | VTLTCVAPLSGVDFQLR |
| Peptide 57 | 1884.000 | 1883.000 | 57 | EIVLTQSPGTLSLSPGER |
| Peptide 65 | 2186.092 | 2185.092 | 65 | LYHSEAFTVNFGDTEEAKK |
| Peptide 67 | 2296.219 | 2295.219 | 67 | TPGAAANLELIFVGPQHAGNYR |
| Peptide 68 | 2315.195 | 2314.195 | 68 | QSNNKYAASSYLSLTPEQWK |
| Peptide 70 | 2348.245 | 2347.245 | 70 | AHVDALRTHLAPYSDELRQR |
| Peptide 75 | 2652.448 | 2651.448 | 75 | TVRTPGAAANLELIFVGPQHAGNYR |
| Peptide 76 | 2670.387 | 2669.387 | 76 | DEPPQSPWDRVKDLATVYVDVLK |
| Peptide 77 | 2695.362 | 2694.362 | 77 | QSNNKYAASSYLSLTPEQWKSHR |
| Peptide 78 | 2790.425 | 2789.425 | 78 | YRSWVPHTFESELSDPVELLVAES |

In another, more preferred embodiment, the peptide pattern of the invention further comprises at least one peptide selected from the group consisting of the peptides shown in Table 4, which are characterized by both their specific amino acid sequence, and their mass/charge value and molecular weight. The peptides shown in Table 4 may be used in combination with the peptide pattern of the invention in order to differentiate between arthrotic subjects and normal subjects. In another preferred embodiment, arthrotic subjects show a decreased presence of the peptides shown in Table 4 as compared to the healthy control subjects.

TABLE 4
Sequences and mass/charge values and molecular
weights obtained by means of mass spectrometry
for each additional peptide that may be used in
combination with the peptide pattern of the
invention in order to differentiate between
arthrotic subjects and normal subjects.

| | Mass/ | Molecular | SEQ | |
|---|---|---|---|---|
| | charge | weight | ID | |
| | (m/z) | (Da) | NO: | Peptide sequence |
| Peptide 12 | 871.502 | 870.502 | 12 | VLNQELR |
| Peptide 34 | 1518.71 | 1517.71 | 34 | ADSGEGDFLAEGGGVR |
| Peptide 50 | 1810.992 | 1809.992 | 50 | SYFEKSKEQLTPLIK |
| Peptide 60 | 1922.877 | 1921.877 | 60 | SEAEDASLLSFMQGYMK |
| Peptide 62 | 2012.011 | 2011.011 | 62 | TFGSGEADCGLRPLFEKK |
| Peptide 64 | 2153.112 | 2152.112 | 64 | SLEDKTERELLESYIDGR |
| Peptide 74 | 2602.358 | 2601.358 | 74 | AVPPNNSNAAEDDLPTVE |
| | | | | LQGVVPR |

In another particular embodiment, the peptide pattern of the invention further comprises at least one peptide selected from the group consisting of the peptides shown in Table 5, which are characterized by both their specific amino acid sequence, and their mass/charge value and molecular weight. The peptides shown in Table 5 may be used in combination with the peptide pattern of the invention in order to differentiate between arthrotic subjects and subjects suffering from rheumatoid arthritis.

TABLE 5
Sequences, mass/charge values and molecular
weights obtained by means of mass spectrometry
for each additional peptide that may be used in
combination with the peptide pattern of the
invention in order to differentiate between
arthrotic subjects and subjects suffering from
rheumatoid arthritis.

| | Mass/ | Molecular | SEQ | |
|---|---|---|---|---|
| | charge | weight | ID | Peptide |
| | (m/z) | (Da) | NO: | sequence |
| Peptide 22 | 1247.652 | 1246.652 | 22 | LGMFNIQHCK |
| Peptide 23 | 1249.655 | 1248.655 | 23 | LICQATGFSPR |
| Peptide 26 | 1299.609 | 1298.609 | 26 | WQEEMELYR |
| Peptide 30 | 1465.689 | 1464.689 | 30 | DSGEGDFLAEGGGVR |
| Peptide 31 | 1467.852 | 1466.852 | 31 | VEPLRAELQEGAR |
| Peptide 35 | 1519.75 | 1518.75 | 35 | ALYLQYTDETFR |
| Peptide 39 | 1612.849 | 1611.849 | 39 | LLDNWDSVTSTFSK |
| Peptide 45 | 1736.97 | 1735.97 | 45 | LSQRFPKAEFAEVSK |
| Peptide 46 | 1743.882 | 1742.882 | 46 | YAASSYLSLTPEQWK |
| Peptide 47 | 1745.901 | 1744.901 | 47 | YVGGQEHFAHLLILR |
| Peptide 48 | 1771.885 | 1770.885 | 48 | SLAELGGHLDQQVEEF |
| Peptide 58 | 1899.060 | 1898.060 | 58 | FNKPFVFLMIEQNTK |
| Peptide 59 | 1906.873 | 1905.873 | 59 | SEAEDASLLSFMQGYMK |
| Peptide 61 | 1932.038 | 1931.038 | 61 | SLHTLFGDKLCTVATLR |
| Peptide 63 | 2020.138 | 2019.138 | 63 | VLDAVRGSPAINVAVHV |
| | | | | FR |
| Peptide 66 | 2190.132 | 2189.132 | 66 | CEGPIPDVTFELLREGE |
| | | | | TK |
| Peptide 69 | 2344.169 | 2343.169 | 69 | SEAEDASLLSFMQGYMK |
| | | | | HATK |
| Peptide 71 | 2394.298 | 2393.298 | 71 | LTPYADEFKVKIDQTVE |
| | | | | ELR |
| Peptide 72 | 2409.281 | 2408.281 | 72 | GAHGPRPDTVGQRGGSR |
| | | | | PSPGPIR |
| Peptide 73 | 2565.322 | 2564.322 | 73 | GPPGLPGLKGDPGVPGL |
| | | | | PGAKGEVGADGV |

In another particular embodiment, the peptide pattern of the invention further comprises at least one peptide selected from the group consisting of the peptides shown in Table 6, which are characterized by both their specific amino acid sequence, and their mass/charge value and molecular weight. The peptides shown in Table 6 may be used in combination with the peptide pattern of the invention in order to differentiate between arthrotic subjects and subjects suffering from psoriatic arthritis.

TABLE 6
Sequences, mass/charge values and molecular
weights obtained by means of mass spectrometry
for each additional peptide that may be used in
combination with the peptide pattern of the
invention in order to differentiate between
arthrotic subjects and subjects suffering from
psoriatic arthritis.

| | Mass/ | Molecular | SEQ | |
|---|---|---|---|---|
| | charge | weight | ID | Peptide |
| | (m/z) | (Da) | NO: | sequence |
| Peptide 22 | 1247.652 | 1246.652 | 22 | LGMFNIQHCK |
| Peptide 25 | 1263.683 | 1262.683 | 25 | LGMFNIQHCK |
| Peptide 26 | 1299.609 | 1298.609 | 26 | WQEEMELYR |
| Peptide 27 | 1380.78 | 1379.78 | 27 | VQPYLDDFQKK |
| Peptide 29 | 1449.8 | 1448.8 | 29 | VVAGVANALAHKYH |
| Peptide 30 | 1465.689 | 1464.689 | 30 | DSGEGDFLAEGGGVR |
| Peptide 31 | 1467.852 | 1466.852 | 31 | VEPLRAELQEGAR |
| Peptide 37 | 1569.907 | 1568.907 | 37 | LAARLEALKENGGAR |
| Peptide 39 | 1612.849 | 1611.849 | 39 | LLDNWDSVTSTFSK |
| Peptide 46 | 1743.882 | 1742.882 | 46 | YAASSYLSLTPEQWK |
| Peptide 47 | 1745.901 | 1744.901 | 47 | YVGGQEHFAHLLILR |
| Peptide 48 | 1771.885 | 1770.885 | 48 | SLAELGGHLDQQVEEF |
| Peptide 49 | 1786.909 | 1785.909 | 49 | GLPGPPDVPDHAAYHPF |
| Peptide 58 | 1899.060 | 1898.060 | 58 | FNKPFVFLMIEQNTK |
| Peptide 59 | 1906.873 | 1905.873 | 59 | SEAEDASLLSFMQGYMK |
| Peptide 66 | 2190.132 | 2189.132 | 66 | CEGPIPDVTFELLREGETK |
| Peptide 69 | 2344.169 | 2343.169 | 69 | SEAEDASLLSFMQGYMKHA |
| | | | | TK |
| Peptide 79 | 2940.755 | 2939.755 | 79 | SASLHLPKLSITGTYDLKS |
| | | | | VLGQLGITK |

The methods and techniques used to obtain peptide patterns from a sample are widely known in the state of the art, and will be explained in detail in the description of the diagnostic method of the invention.

Another aspect of the invention relates to the use of the peptide pattern of the invention as a diagnostic marker for arthrosis in a subject.

Thus, another object of the present invention relates to the simultaneous in vitro use of the expression level of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11, as a marker designed to obtain useful data for the diagnosis and classification of rheumatic diseases in an subject.

In a preferred embodiment, the in vitro use of the peptide pattern of the invention is characterized in that it further comprises determining the expression level of at least one of the peptides selected from the list consisting of: SEQ ID NO: 12; SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 34, SEQ ID NO: 36, SEQ ID NO: 38, SEQ ID NO: 40, SEQ ID NO: 41, SEQ ID NO: 42, SEQ ID NO: 43, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 60, SEQ ID NO: 62, SEQ ID NO: 64, SEQ ID NO: 65, SEQ ID NO: 67, SEQ ID NO: 68, SEQ ID NO: 70, SEQ ID NO: 74, SEQ ID NO: 75, SEQ ID NO: 76, SEQ ID NO: 77, SEQ ID NO: 78, SEQ ID NO: 22, SEQ ID NO: 23, SEQ ID NO: 26, SEQ ID NO: 30, SEQ ID NO: 31, SEQ ID NO: 35, SEQ ID NO: 39, SEQ ID NO: 45, SEQ ID NO: 46, SEQ ID NO: 47, SEQ ID NO: 48, SEQ ID NO: 58, SEQ ID NO: 59, SEQ ID NO: 61, SEQ ID NO: 63, SEQ ID NO: 66, SEQ ID NO: 69, SEQ ID NO: 71, SEQ ID NO: 72, SEQ ID NO: 73, SEQ ID NO: 25, SEQ ID NO: 27, SEQ ID NO: 29, SEQ ID NO: 37, SEQ ID NO: 49, SEQ ID NO: 79, or combinations thereof.

In another preferred embodiment, the in vitro use of the peptide pattern of the invention is characterized in that the rheumatic diseases are selected from any of the following: arthrosis, rheumatoid arthritis and psoriatic arthritis.

Another aspect of the present invention relates to a method for obtaining useful data, hereinafter called first method of the invention, for the diagnosis and classification of rheumatic diseases, which comprises:

(a) obtaining an isolated biological sample from a subject,
(b) simultaneously detecting, in the isolated biological sample of (a), the expression level of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11.

In a preferred embodiment, the first method of the invention further comprises determining, in step (b), the expression level and/or the quantity of at least one of the peptides selected from the list consisting of: SEQ ID NO: 12; SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 34, SEQ ID NO: 36, SEQ ID NO: 38, SEQ ID NO: 40, SEQ ID NO: 41, SEQ ID NO: 42, SEQ ID NO: 43, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 60, SEQ ID NO: 62, SEQ ID NO: 64, SEQ ID NO: 65, SEQ ID NO: 67, SEQ ID NO: 68, SEQ ID NO: 70, SEQ ID NO: 74, SEQ ID NO: 75, SEQ ID NO: 76, SEQ ID NO: 77, SEQ ID NO: 78, SEQ ID NO: 22, SEQ ID NO: 23, SEQ ID NO: 26, SEQ ID NO: 30, SEQ ID NO: 31, SEQ ID NO: 35, SEQ ID NO: 39, SEQ ID NO: 45, SEQ ID NO: 46, SEQ ID NO: 47, SEQ ID NO: 48, SEQ ID NO: 58, SEQ ID NO: 59, SEQ ID NO: 61, SEQ ID NO: 63, SEQ ID NO: 66, SEQ ID NO: 69, SEQ ID NO: 71, SEQ ID NO: 72, SEQ ID NO: 73, SEQ ID NO: 25, SEQ ID NO: 27, SEQ ID NO: 29, SEQ ID NO: 37, SEQ ID NO: 49, SEQ ID NO: 79, or combinations thereof.

In another preferred embodiment, the first method of the invention is characterized in that the rheumatic diseases are selected from any of the following: arthrosis, rheumatoid arthritis and psoriatic arthritis.

Another aspect of the present invention relates to a diagnostic and classification method for arthrotic diseases, hereinafter called second method of the invention, which comprises steps (a) and (b) according to the first method of the invention, and further comprises:

(c) comparing the quantities obtained in step (b) for the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 34, SEQ ID NO: 36, SEQ ID NO: 38, SEQ ID NO: 40, SEQ ID NO: 41, SEQ ID NO: 42, SEQ ID NO: 43, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 60, SEQ ID NO: 62, SEQ ID NO: 64, SEQ ID NO: 65, SEQ ID NO: 67, SEQ ID NO: 68, SEQ ID NO: 70, SEQ ID NO: 74, SEQ ID NO: 75, SEQ ID NO: 76, SEQ ID NO: 77 and SEQ ID NO: 78, with a reference quantity from a sample obtained from a healthy control subject, and

(d) assigning the subject of step (a) to the group of subject suffering from arthrosis when they present:
(i) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, detected in (b), or
(ii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
(iii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77, and SEQ ID NO: 78, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b),
as compared to the expression level and/or the quantity of the same peptides detected in a reference sample obtained from a healthy control subject.

Another object of the present invention relates to a diagnostic and classification method for arthrotic diseases, hereinafter third method of the invention, which comprises steps (a) and (b) according to the first method of the invention and further comprises:
(c) comparing the quantities obtained in step (b) for the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 34, SEQ ID NO: 50, SEQ ID NO: 60, SEQ ID NO: 62, SEQ ID NO: 64 and SEQ ID NO: 74, SEQ ID NO: 22, SEQ ID NO: 23, SEQ ID NO: 26, SEQ ID NO: 31, SEQ ID NO: 35, SEQ ID NO: 39, SEQ ID NO: 45, SEQ ID NO: 46, SEQ ID NO: 47, SEQ ID NO: 48, SEQ ID NO: 58, SEQ ID NO: 61, SEQ ID NO: 63, SEQ ID NO: 66, SEQ ID NO: 71, SEQ ID NO: 72 and SEQ ID NO: 73, SEQ ID NO: 30, SEQ ID NO: 59 and SEQ ID NO: 69, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, with a reference quantity from a sample obtained from a subject suffering from arthrosis, and
(d) assigning the subject of step (a) to the group of subjects suffering from rheumatoid arthritis when they present:
   (i) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6 SEQ ID NO: 7, SEQ ID NO: 8, SEQ ID NO: 9, SEQ ID NO: 10, SEQ ID NO: 11, SEQ ID NO: 22, SEQ ID NO: 23, SEQ ID NO: 26, SEQ ID NO: 31, SEQ ID NO: 35, SEQ ID NO: 39, SEQ ID NO: 45, SEQ ID NO: 46, SEQ ID NO: 47, SEQ ID NO: 48, SEQ ID NO: 58, SEQ ID NO: 61, SEQ ID NO: 63, SEQ ID NO: 66, SEQ ID NO: 71, SEQ ID NO: 72 and SEQ ID NO: 73, detected in (b); or
   (ii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and a significantly lower expression level and/or quantity of the peptides with the sequences SEQ ID NO: 30, SEQ ID NO: 59 and SEQ ID NO: 69, detected in (b); or
   (iii) a significantly higher expression level and/or quantity of the peptides of step (i), and the peptides with the sequences SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, detected in (b); or
   (iv) a significantly higher expression level and/or quantity of the peptides of step (i), and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
   (v) a significantly higher expression level and/or quantity of the peptides of step (iii), and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
   (vi) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, and a significantly lower expression level and/or quantity of the peptides with the sequences SEQ ID NO: 30, SEQ ID NO: 59 and SEQ ID NO: 69, detected in (b); or
   (vii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, SEQ ID NO: 30, SEQ ID NO: 59 and SEQ ID NO: 69, detected in (b); or
   (viii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64, SEQ ID NO: 74, SEQ ID NO: 30, SEQ ID NO: 59 and SEQ ID NO: 69,
   as compared to the expression level and/or the quantity of the same peptides detected in a reference sample obtained from a subject suffering from arthrosis.

Another aspect of the present invention relates to a diagnostic and classification method for arthrotic diseases, hereinafter called fourth method of the invention, which comprises steps (a) and (b) according to the first method of the invention, and further comprises:
(c) comparing the quantities obtained in step (b) for the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 22, SEQ ID NO: 26, SEQ ID NO: 27, SEQ ID NO: 31, SEQ ID NO: 37, SEQ ID NO: 39, SEQ ID NO: 46, SEQ ID NO: 47, SEQ ID NO: 48, SEQ ID NO: 49, SEQ ID NO: 58, SEQ ID NO: 66 and SEQ ID NO: 79, SEQ ID NO: 25, SEQ ID NO: 29, SEQ ID NO: 30, SEQ ID NO: 59 and SEQ ID NO: 69, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, SEQ ID NO: 12, SEQ ID NO: 34, SEQ ID NO: 50, SEQ ID NO: 60, SEQ ID NO: 62, SEQ ID NO: 64 and SEQ ID NO: 74, with a reference quantity from a sample obtained from a subject suffering from arthrosis, and
d) assigning the subject of step (a) to the group of subjects suffering from psoriatic arthritis when they present:
   (i) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 22, SEQ ID NO: 26, SEQ ID NO: 27, SEQ ID NO: 31, SEQ ID NO: 37, SEQ ID NO: 39, SEQ ID NO: 46, SEQ ID NO: 47, SEQ ID NO: 48, SEQ ID NO: 49, SEQ ID NO: 58, SEQ ID NO: 66 and SEQ ID NO: 79, detected in (b); or
   (ii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11, and a significantly lower expression level and/or quantity of the peptides with the sequences SEQ ID NO: 25, SEQ ID NO: 29, SEQ ID NO: 30, SEQ ID NO: 59 and SEQ ID NO: 69, detected in (b); or
   (iii) a significantly higher expression level and/or quantity of the peptides of step (i), and the peptides with the sequences SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, detected in (b); or
   (iv) a significantly higher expression level and/or quantity of the peptides of step (i), and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
   (v) a significantly higher expression level and/or quantity of the peptides of step (iii), and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
   (vi) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, and a significantly lower expression level and/or quantity of the peptides with the sequences SEQ ID NO: 25, SEQ ID NO: 29, SEQ ID NO: 30, SEQ ID NO: 59 and SEQ ID NO: 69, detected in (b); or
   (vii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, SEQ ID NO: 25, SEQ ID NO: 29, SEQ ID NO: 30, SEQ ID NO: 59 and SEQ ID NO: 69, detected in (b); or
   (viii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, SEQ ID NO: 25, SEQ ID NO: 29, SEQ ID NO: 30, SEQ ID NO: 59 and SEQ ID NO: 69, detected in (b);
   as compared to the expression level and/or the quantity of the same peptides detected in a reference sample from a group of subjects suffering from arthrosis.

The reference quantity is obtained from the expression values and/or constitutive quantities of the peptide(s) described in the present invention for a group of healthy subjects or individuals, or, preferably, who do not present an arthrotic disease, or from a group of subjects who present an arthrotic disease, according to each of the methods described above and throughout the present document. The reference sample may be analysed, for example, simultaneously or consecutively with the test biological sample. The comparison described in Section (c) of the method of the present invention may be performed manually or with computer assistance.

In a more preferred embodiment of all the methods described in the present invention, these are characterized in that steps (b) and/or (c) may be totally or partially automated, for example, by means of a sensor robotic equipment, for the detection of the quantity of step (b) or the computerised comparison of step (c).

In another more preferred embodiment of all the methods described in the present invention, these are characterized in that the sample is a biological fluid.

In the present invention, “diagnosis” is understood as the process whereby a given disease, nosological entity, syndrome, or any health-disease condition is identified, by analysing a number of clinical parameters or symptoms characteristic of said disease, and which differentiate it from other diseases with similar symptoms. In the present invention, the disease, nosological entity or syndrome to be detected is arthrosis, and the clinical parameter is the peptide pattern of the invention.

In the present invention, “rheumatic diseases” are understood as those disorders that generally affect the locomotor and the musculo-skeletal system, preferably the joints, tendons, ligaments, bones and muscles. For the purposes of the present invention, the rheumatic diseases are selected from any of the following: arthrosis, rheumatoid arthritis and psoriatic arthritis.

For the purposes of the present invention, the term “arthrosis” or “osteoarthritis” refers to a chronic articular disease caused by deterioration of the hyaline cartilage and osteoblastic hyperactivity of subchondral bone. In the present invention, the terms “arthrosis” and “osteoarthritis” are equivalent and may be used interchangeably throughout the description thereof. As known to persons skilled in the art, arthrosis may be classified into idiopathic arthrosis and secondary arthrosis (modified from Altman R C, Semin Arthritis Rheum 1991; 20:40-47). Idiopathic arthrosis (the origin of the disease is unknown) may be localised, for example in the hands, feet, hips, spine, etc., or generalised, in which case three or more articular areas are affected, such as the small joints and the spine, the large joints and the spine, or mixed, which is a combination of the above. Secondary arthrosis is classified into congenital or developmental diseases (including, without being limited thereto, Gaucher's disease, the Legg-Calvé disease, bone dysplasias, etc.), calcium deposition diseases (which include, without being limited thereto, hydroxyapatite arthropathy, destructive arthropathy and calcium pyrophospate deposition arthropathy), post-traumatic diseases, bone and articular diseases (which include, without being limited thereto, avascular necrosis, rheumatoid arthritis, gouty arthritis, septic arthritis, Paget's disease, osteopetrosis, osteochondritis), and various diseases originating from arthritis (which include, without being limited thereto, Charcot joint, diabetes mellitus, acromegaly, hypothyroidism, hyperparathyroidism, Kashin-Beck disease, Caisson's disease, etc.). On the other hand, arthrosis may also be classified using the radiographic KELLGREN-LAWRENCE (K/L) scale, which classifies arthrosis into 5 radiographic grades: Grades 0, I, II, III, and IV.

Any of the diseases included within the classification of arthrotic diseases, including arthrosis, rheumatoid arthritis and psoriatic arthritis, may be diagnosed and classified using the peptide pattern of the invention.

For purposes of the present invention, the term “rheumatoid arthritis (RA)” refers to an inflammatory disease of unknown etiology, characterized by affectation of the diarthrodial joints and the development of chronic synovitis, which progressively leads to articular damage, functional impairment and a significant deterioration of the patients' quality of life. In addition to articular affectation, RA is also characterized by a sustained systemic inflammatory process and the possibility of developing severe extra-articular manifestations, which often determine the prognosis of the disease.

For purposes of the present invention, the term “psoriatic arthritis (PsA)” refers to an articular disease that appears in some patients (approximately 10%) who suffer from psoriasis of the skin, which gives it peculiar characteristics in terms of evolution and prognosis. The articular injury is inflammatory, i.e. is accompanied by pain, swelling, warmth, difficulty moving the inflamed joint and, in the long term, the possibility of deformation. It is a chronic disease, which evolves in an irregular manner throughout life, with periods of inactivity and periods of inflammation and pain.

In the present invention, “subject” or “individual” is understood to mean any animal, preferably a mammal, more preferably a primate, and, in particular, a human being, of any race, sex, age or physical condition. However, in a particular embodiment, the subject is a woman; more particularly, a woman older than 50.

The implementation of the methods of the invention described herein comprises determining the expression level and/or the concentration or quantity of the peptides of the invention; preferably, it comprises determining the presence or absence of the peptides that comprise the peptide pattern of the invention in a biological sample from said subject or individual.

In the present invention, “biological sample” is understood to mean any material of animal origin, in particular of human origin, including excreta (faeces and urine), secretions (genital, respiratory, etc.), blood or the components thereof, organic tissues and fluids (biopsies, fluids: cephalo-rachidian, synovial, articular, ascitic, etc.), etc. In a particular embodiment of the methods of the invention, the sample taken from the subject is a biological fluid, which, in another, even more particular embodiment, is selected from the group consisting of blood, serum, plasma, synovial fluid and urine; more preferably, serum, plasma and synovial fluid.

Once the biological sample has been obtained from the subject, it must be processed to obtain the peptides described in the present invention. Thus, the biological sample may be treated such that the structure of the tissue or cell is physically or mechanically broken down and releases the intracellular components into an aqueous or organic solution. Once this has been performed, the proteins are isolated from the sample. Any of the techniques known in the state of the art to isolate proteins may be used in the present invention, such as, for example, centrifugation, depletion, etc. Once the proteins have been isolated, they are digested in order to obtain the peptide profile of the invention.

Once the peptide fraction has been obtained, and depending on the technique used to analyse the expression, the protein fraction obtained may or may not be digested with trypsin, to perform enzymatic digestion of the proteins and obtain peptides. Trypsin is a peptidase enzyme that breaks down the peptide bonds within proteins by means of hydrolysis, to form smaller-size peptides and amino acids. Trypsin is produced in the pancreas and secreted in the duodenum (part of the intestine), where it is essential for digestion. The optimal pH is 8 and the optimal temperature is 37° C. For purposes of the present invention, any technique known and used in the state of the art to isolate the peptide fraction from a biological sample may be used in the present invention.

In another more preferred embodiment, the methods of the invention described herein are characterized in that the detection of the expression levels and/or the quantity or concentration of the peptides is performed by means of techniques that are widely known in the state of the art. Examples of said techniques include, without being limited thereto, mass spectrometry, gas chromatography-mass spectrometry or liquid chromatography-mass spectrometry, flow cytometry, Western Blot or Western transfer, ELISA (enzyme-linked immunosorbent assay), RIA (radioimmunoassay), competitive EIA (competitive enzyme immunoassay), DAS-ELISA (double-antibody sandwich ELISA) and immunocytochemical and immunohistochemical techniques.

Mass spectrometry is an analytical technique that makes it possible to elucidate chemical structures by measuring the mass/charge ratio of molecular species. The determinations require generating electrically charged species, which is performed by means of different methodologies, such as electron impact, fast atom bombardment (FAB) and generation of bound ions. The measurement of the mass/charge ratio makes it possible to determine the exact molecular weight of the molecule. Other techniques derived from mass spectrometry that may also be used within the context of the present invention include, without being limited thereto, gas chromatography-mass spectrometry (GC-MS) and liquid chromatography-mass spectrometry (LC-MS).

The ELISA technique (acronym for Enzyme-Linked ImmunoSorbent Assay) is an immunoassay technique wherein an immobilised antigen is detected by means of an enzyme-linked antibody capable of generating a detectable product, such as a change in colour or any other type.

In the present invention, the term “antibody” is broadly interpreted to include polyclonal, monoclonal and multi-specific antibodies, and fragments thereof [F(ab′)2, Fab, scFv, etc.], provided that they are capable of recognising the antigen of interest, i.e. of binding to the peptides comprised within the peptide pattern of the invention. Examples of antibodies that may be used within the context of the present invention include, without being limited thereto, polyclonal antibodies, monoclonal antibodies, recombinant antibodies, chimeric antibodies, humanised antibodies, totally human antibodies, etc. The antibodies used in these assays may or may not be labelled. Illustrative examples of labels that may be used include radioactive isotopes, enzymes, fluorophores, chemiluminescent reagents, enzyme substrates or co-factors, enzyme inhibitors, particles, dyes, etc.

Flow cytometry is a biophysical technology based on the use of laser light, wherein the proteins, which are bound to a labelled antibody and suspended in a fluid, go through a very thin transparent tube that is hit by a thin laser beam; the light transmitted and dispersed when the proteins go through the tube is collected by means of detection devices, making it possible to detect and separate the proteins bound to the labelled antibodies.

The detection of the quantity of the peptides described throughout the present document may be performed by any means known in the state of the art. Thus, the detection of the levels or quantities of said peptides may be performed in a semi-quantitative or quantitative manner, which makes it possible to differentiate between the different types of subjects, healthy subjects and those suffering from arthrosis, rheumatoid arthritis or psoriatic arthritis. In this way, a differential diagnosis may be made for the subjects affected by the aforementioned diseases, which makes it possible to sub-classify them.

The measurement of the quantity or concentration of these peptides, preferably in a semi-quantitative or quantitative manner, may be performed directly or indirectly. Direct measurement refers to measuring the quantity or concentration of the expression product thereof, based on a signal that is directly obtained from the expression of said peptides, and is directed correlated to the number of proteins present in the samples analysed. Said signal—which may also be called intensity signal—may be obtained, for example, by measuring the intensity value of a chemical or physical property of said products. Indirect measurement includes the measurements obtained from a secondary component or a biological measurement system (for example, measurement of cellular responses, ligands, “labels” or enzymatic reaction products).

As used in the description, the term “quantity” refers, without being limited thereto, to the absolute or relative quantity of the peptides or the molecules capable of detecting them, as well as to any other value or parameter related to them or which may be derived from them. Said values or parameters comprise signal intensity values obtained for any physical or chemical property of said expression products by means of direct measurement. Moreover, said values or parameters include all those obtained by means of indirect measurement, for example, any of the measurement systems described elsewhere in the present document.

Another aspect of the invention relates to a kit or device, hereinafter “kit of the invention”, which comprises the necessary molecular tools to detect the expression level and/or quantity of the peptides described in the present invention, or any combination thereof, for the diagnosis and classification of rheumatic diseases.

In a more preferred embodiment, the kit of the invention comprises the necessary molecular tools to detect the expression level and/or quantity of the peptides with the sequences SEQ ID NO: 1 to 11.

In another, even more preferred embodiment, the kit of the invention further comprises the necessary molecular tools to detect the expression level and/or quantity of at least one of the peptides with the sequences: SEQ ID NO: 12; SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 34, SEQ ID NO: 36, SEQ ID NO: 38, SEQ ID NO: 40, SEQ ID NO: 41, SEQ ID NO: 42, SEQ ID NO: 43, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 60, SEQ ID NO: 61, SEQ ID NO: 62, SEQ ID NO: 64, SEQ ID NO: 65, SEQ ID NO: 66, SEQ ID NO: 67, SEQ ID NO: 68, SEQ ID NO: 70, SEQ ID NO: 74, SEQ ID NO: 75, SEQ ID NO: 76, SEQ ID NO: 77, SEQ ID NO: 78, SEQ ID NO: 22, SEQ ID NO: 26, SEQ ID NO: 30, SEQ ID NO: 31, SEQ ID NO: 35, SEQ ID NO: 39, SEQ ID NO: 45, SEQ ID NO: 46, SEQ ID NO: 47, SEQ ID NO: 48, SEQ ID NO: 58, SEQ ID NO: 59, SEQ ID NO: 61, SEQ ID NO: 63, SEQ ID NO: 66, SEQ ID NO: 69, SEQ ID NO: 71, SEQ ID NO: 72, SEQ ID NO: 73, SEQ ID NO: 25, SEQ ID NO: 27, SEQ ID NO: 29, SEQ ID NO: 37, SEQ ID NO: 49, SEQ ID NO: 69, SEQ ID NO: 79, or combinations thereof.

In a more preferred embodiment of the kit, said molecules capable of detecting the expression levels and/or quantities of the peptides described in the present invention are preferably selected from any of the following: specific antibodies against the peptides that comprise the peptide pattern of the invention or fragments thereof, and oligonucleotides capable of hybridising to the nucleotide sequence that encodes the peptides described in the present invention.

In a preferred embodiment, said oligonucleotides are designed from a nucleotide sequence that encodes the peptides described in the present document.

In a preferred embodiment, the kit of the invention is useful for the diagnosis and classification of rheumatic diseases in a subject or individual, wherein said rheumatic diseases are selected from any of the following: arthrosis, rheumatoid arthritis and psoriatic arthritis.

The terms “peptide pattern”, “peptides” and “antibody” have already been described and explained in the preceding section, which is applicable to the present inventive aspect.

In another preferred embodiment, the kit of the invention comprises secondary antibodies or positive and/or negative controls. Moreover, the kit may include, without any type of limitation, buffers, protein extraction solutions, contamination prevention agents, protein degradation inhibitors, etc. On the other hand, the kit may include all the necessary supports and containers for the implementation and optimisation thereof. Preferably, the kit further comprises instructions for performing the methods of the invention.

Within the context of the present invention, “kit” is understood to mean a product that contains the different reagents needed to implement the method of the invention, packed in order to allow for the transport and storage thereof. Suitable materials for packing the kit components include, without being limited thereto, glass, plastic (polyethylene, polypropylene, polycarbonate and similar materials), bottles, vials, paper, sachets and similar materials.

Furthermore, the kit may contain instructions for using the different kit components. Said instructions may be in the form of printed material or in the form of an electronic medium capable of storing instructions such that they may be read by a subject, such as electronic storage media (magnetic disks, tapes and similar media), optical media (CD-ROM, DVD) and similar media. Additionally or alternatively, the media may contain Internet addresses that supply said instructions.

Another aspect of the present invention relates to the use of the kit of the invention to obtain useful data for the diagnosis and classification of rheumatic diseases, preferably arthrosis, rheumatoid arthritis and psoriatic arthritis, from a biological sample. In a more preferred embodiment, the biological sample is a biological fluid, which is preferably selected from the group consisting of blood, serum, plasma, synovial fluid and urine.

Throughout the description and the claims, the word “comprises” and variants thereof are not intended to exclude other technical characteristics, additives, components or steps. For persons skilled in the art, other objects, advantages and characteristics of the invention will arise, partly from the description and partly from the implementation of the invention.

### BRIEF DESCRIPTION OF THE FIGURES

FIG. 1: Diagrams of the principal component analyses (PCA) performed with the peptides of the invention and obtained by means of the MALDI-TOF technique, for the group of arthrotic patients (grey) and for the group of control patients (black), at each elution percentage. The PCAs show that, under all the extraction conditions, classification of the samples is achieved in at least two planes.

### EXAMPLES

The following examples and FIGURES are provided for illustrative purposes, and are not intended to limit the scope of the present invention.

### Example 1. Material and Methods

### 1.1 Reagents

### DL-dithiothreitol (DTT≧99%)

### Iodoacetamide (IAA≧99%)

Trifluoroacetic acid (TFA≧99%, for LC-MS)

### Acetonitrile (ACN, LC-MS CHROMASOLV)

### Water (LC-MS CHROMASOLV)

Ammonium bicarbonate≧99%)
Trypsin (sequencing grade, Roche)

### Large NuTips, 10-200 μl, C-18 (Glygen, Columbia, USA)

α-Cyano-4-hydroxycinnamic acid, ultra-pure, for MALDI-MS (Fluke, Germany)
Calibration mixture 1, 4700 Proteomics Analyzer (ABSciex, Framingham, Mass., USA)

### 1.2. Serum Samples

The human serum samples were obtained from anonymous donors at the A Coruña University Hospital Complex, Spain. All the patients signed the informed consent. The study was approved by the Local Ethics Committee of Galicia (Spain). The following were selected: 40 serum samples from arthrotic donors (OA), 40 serum samples from donors diagnosed with rheumatoid arthritis (RA), 40 samples from donors diagnosed with psoriatic arthritis (PsA), and 40 serum samples from donors without any of the aforementioned pathologies, i.e. subjects who did not present any type of rheumatic pathology. The latter group is the control group (N). 20 sera from each group of patients were used to search for the peptide pattern characteristic of each pathology. Subsequently, the other remaining 20 sera from each group of patients were used as blind samples in order to verify the validity of the peptide pattern of the invention. The serum samples were stored at −80° C. until the processing thereof.

### 1.3. Sequential Serum Protein Depletion

Protein depletion was performed according to the protocol that was previously developed at our laboratory (Fernandez-Costa et al., Proteome Sci 2012; 55). Each of the sera were subjected to a sequential depletion protocol involving two precipitation steps: first with DTT and, subsequently, with ACN, according to the protocol described by Kay et al. (Rapid Commun Mass Spectrom, 2008, 22: 3255-60), with slight modifications. Briefly, 2.2 μl of 500 mM DTT were added to 20 μl of serum and vortex-stirred. Thereafter, the sample was incubated for 1 hour until a persistent white viscous precipitate was formed, which was sedimented by centrifugation at 14,000×g for 2×20 minutes. The supernatant was transferred to a clean tube (LoBind, Eppendorf) and depleted once again by the addition of 57% (v/v) acetonitrile, followed by two short vortex cycles and 10 minutes of ultrasound in an ultrasonic bath (SONOREX, Bandelin). The protein precipitate was sedimented by centrifugation at 14,000×g for 10 minutes, and the supernatant was transferred to a clean LoBind tube and evaporated to dryness using a vacuum centrifuge.

### 1.4. In-Solution Digestion

The ultrasonic in-solution digestion was performed according to the previously developed ultra-rapid proteolytic digestion protocol (H M Santos et al. J Proteome Res 2007.6: 3393-9). The evaporated sample was re-suspended in 20 μl of 25 mM ammonium bicarbonate (AmBi), and 10 μl of acetonitrile were added and mixed by means of vortex-mixing and sonication for 1 minute (50% amplitude) in an ultrasonic bath (SONOPULS HD 2200 with BB6 accessory, Bandelin). The protein cysteine residues were reduced with 2 μl of 110 mM DTT, followed by vortex-mixing and 1 minute of sonication, and, subsequently, blocked with 2 μl of 600 mM IAA, vortex-mixing and 1 minute of sonication. In order to de-activate the IAA, 10 μl of 110 mM DTT were added to the sample. Thereafter, the sample was diluted to a final volume of 200 μl with 12.5 mM AmBi, and trypsin was added at a 1:20 (w/w) trypsin:protein ratio. The digestion was performed in the ultrasonic equipment for 5 minutes with a 50% amplitude. Finally, 2 μl of 50% (v/v) formic acid were added in order to stop the enzymatic reaction. The digested serum was evaporated to dryness in a vacuum centrifuge.

### 1.5. Sequential Elution by Means of NuTips (Peptide Separation)

The digested samples were reconstituted in 30 μl of 0.1% (v/v) TFA, and 20 μg of the digest were loaded onto a NuTip C18 (large) type tip. The peptide separation was performed according to the following protocol:

### Conditioning:

30 μl of a solution of 60%-80% (v/v) ACN and 0.1% (v/v) TFA were aspirated and expelled 5 times, and washed 3 times with 0.1% TFA.

### Binding:

the sample was aspirated and expelled 50 times, in order to allow for the peptides to adsorb to the reversed-phase material packed in the tip.

### Sequential Elution:

30 μl of different percentages of ACN, from low to high concentrations of ACN, were aspirated and expelled (20 times each). The sequential elution was performed with 4%, 7%, 10% and 14% (v/v) ACN. The eluates were evaporated to dryness.

### 1.6. Analysis by Means of Mass Spectrometry

The evaporated samples were re-suspended in 6 μl of 0.1% (v/v) TFA, and 1 μl of each sample was deposited in quintuplicate on a MALDI plate (384-well plate coated with Teflon®), and allowed to air-dry at room temperature. Subsequently, 1 μl of a solution of 3 mg/ml of α-cyano-4-hydroxycinnamic acid in 0.1% (v/v) TFA and 50% (v/v) ACN was added to the dry peptide samples, and allowed to dry once again.

The samples were analysed in positive-ion mode using a 4800 Proteomics Analyzer MALDI-TOF/TOF mass spectrometer (ABSciex, Framingham, Mass., USA), equipped with an Nd:YAG laser (k=355 nm) and the 4000 Series Explorer™ programme (ABSciex). The internal calibration, data acquisition, processing and interpretation were performed following the manufacturer's recommendations. All the mass spectra must be externally calibrated using a standard peptide mixture (ABSciex). The mass data were exported on the basis of the following characteristics: a) mass range: 500-4000 (Da); b) density of peaks: maximum 50 peaks per 200 Da; c) signal-to-noise ratio: minimum 20; d) minimum surface area: 100; e) maximum peaks/spot: 500.

The analysis of the mass spectrum for each sample corresponds to an average 1,500 laser pulses. The fragmentation spectra were acquired by selecting the precursors of interest in each MALDI-TOF mass map, with an average 2,000 laser pulses per spectrum. For the fragmentation (MS/MS), all the peaks with a signal-to-noise ratio (S/N)>10 were included in order to perform the database search.

The peptide sequencing was performed using the ProteinPilot™ software (ABSciex), with the following parameters: (i) Version UniProt: 2013_09, which contains 540,958 sequence entries, (ii) Taxonomy: Homo sapiens, (iii) Enzyme: trypsin or without enzyme; (v) Fixed modifications: iodoacetamide. Only those peptides identified with at least 70% confidence were reported.

### 1.7. Bio-Computer Analysis

Each spectrum was pre-processed using the following parameters: mass range between 500 and 4000 Da, maximum density of peaks: 10 peaks per 200 Da, minimum signal-to-noise ratio (S/N) of 10, minimum surface area of 100, maximum number of peaks per spectrum: 120. The peaks were aligned with a peptide mass tolerance of 150 ppm and, finally, a representative spectrum was created for each sample with peaks present in at least 4 of the 5 replica spectra of the sample. For each peak, only their presence or absence in each spectrum was taken into consideration, the use of their intensity beyond pre-processing being discarded.

In order to identify the peaks that were differentially expressed in the different groups, Fisher's test of independence was applied for comparisons between two groups and the Chi-square test of independence was applied for comparisons between the four groups analysed. Since these tests were performed for all the peaks detected, the Benjamini-Hochberg False Discovery Rate (FDR) correction was subsequently applied, in order to reduce the number of false positives.

The separation between the samples under different conditions was graphically analysed using a Principal Component Analysis (PCA).

Finally, in order to verify the utility of the peptides that comprise the peptide pattern described in the present invention for the diagnosis of arthritis, the performance of automatic classifiers trained only with the information from these peptides was evaluated on a set of blind samples.

### Example 2. Results

### 2.1. Obtainment of the Peptide Pattern Characteristic of Arthrosis

In order to obtain the peptide pattern described in the present invention, 20 serum samples from each of the groups of patients included in the present study (OA: arthrosis, RA: rheumatoid arthritis, PsA: psoriatic arthritis, and N: control) were used and, by means of the techniques described in Example 1, the peptides characteristic of each of the study groups that were analysed were detected. Briefly: in first place, the peptides that were significantly detected in one pathology as compared to the others, on the basis of a corrected q value<0.05 for Fisher's test for pairwise comparisons, were selected. Subsequently, those peptides that were significantly detected under two or more conditions on the basis of a corrected q value<0.05 for the Chi-square test of independence, as indicated above, were selected.

By means of said methodology, a total of 11 peptides were identified which were significantly expressed in the group of arthrotic patients as compared to the rest of the groups included in the study. This specific peptide pattern makes it possible to distinguish the samples from those subjects with arthrosis (OA) from the samples from the control subjects (N), and even from the samples of those subjects who suffer from other rheumatic diseases, such as psoriatic arthritis (PsA) and rheumatoid arthritis (RA).

Said set of 11 peptides that make up the peptide pattern of the invention are shown in Table 7.

TABLE 7
Masses of the characteristic peptides that comprise the peptide pattern
for the diagnosis of patients suffering from arthrosis (OA). The
percentages indicate the presence of each peptide in the total samples
analysed for each group of patients, PsA: group of patients suffering
from psoriatic arthritis; RA: group of patients suffering from rheumatoid
arthritis; N: group of control subjects.

| | | PsA | RA | OA | N |
|---|---|---|---|---|---|
| m/z | SEQ ID No. | (n = 20) | (n = 20) | (n = 20) | (n = 20) |
| 1392.7517 | 1 | 0% | 5% | 50% | 10% |
| 1532.796 | 2 | 5% | 15% | 40% | 0% |
| 1758.979 | 3 | 5% | 5% | 40% | 0% |
| 1909.9498 | 4 | 0% | 5% | 40% | 0% |
| 2165.146 | 5 | 10% | 5% | 45% | 0% |
| 2180.1414 | 6 | 15% | 10% | 70% | 20% |
| 2280.1501 | 7 | 5% | 5% | 40% | 5% |
| 2291.227 | 8 | 5% | 10% | 60% | 20% |
| 2407.3042 | 9 | 5% | 0% | 45% | 0% |
| 2436.3015 | 10 | 35% | 25% | 90% | 40% |
| 2663.4382 | 11 | 5% | 5% | 60% | 15% |

The peptides selected as characteristic of arthrosis appear with a significantly greater frequency in the sera of arthrotic donors as compared to the sera of the control patients or patients suffering from PsA or RA.

### 2.2. Additional Specific Peptides for the Diagnosis of Other Pathologies, Such as RA and PsA

In addition to detecting the peptide pattern described in the invention, by means of the methods described above, a set of peptides, in addition to the 11 peptides that comprise the peptide pattern of the invention for the diagnosis of arthrosis, was selected, in order to improve classification of the samples, preferably serum samples, from arthrotic donors. Said additional peptides were selected from the data obtained from the analysis of the 80 serum samples analysed. In this case, since there were comparisons between pairs of conditions, the peptides selected were those for which a corrected q value<0.05 was obtained for Fisher's test.

Table 8 shows the additional peptides that may be used together with the peptide pattern of the invention and which are capable of differentiating between arthrotic subjects and normal subjects who do not suffer from any arthrotic pathology.

TABLE 8
Masses of the additional peptides, other than the peptide pattern
of the invention, useful for classifying and diagnosing patients
suffering from arthrosis (OA), and distinguishing them from
subjects who do not suffer from any arthrotic pathology. The
percentages indicate the presence of each peptide in the total
samples analysed for each group of patients.

| m/z | SEQ ID No. | OA (n = 20) | N (n = 20) |
|---|---|---|---|
| 896.490 | 13 | 65% | 5% |
| 961.627 | 14 | 70% | 15% |
| 980.529 | 15 | 70% | 20% |
| 1019.604 | 16 | 65% | 10% |
| 1108.588 | 17 | 50% | 0% |
| 1117.669 | 18 | 55% | 5% |
| 1148.634 | 19 | 75% | 15% |
| 1213.667 | 20 | 60% | 5% |
| 1237.674 | 21 | 50% | 0% |
| 1255.716 | 24 | 50% | 0% |
| 1403.749 | 28 | 45% | 0% |
| 1470.813 | 32 | 70% | 5% |
| 1502.810 | 33 | 65% | 5% |
| 1568.914 | 36 | 75% | 20% |
| 1585.846 | 38 | 50% | 10% |
| 1641.892 | 40 | 85% | 35% |
| 1641.892 | 41 | 85% | 35% |
| 1669.862 | 42 | 80% | 45% |
| 1706.931 | 43 | 70% | 5% |
| 1815.922 | 51 | 60% | 10% |
| 1825.005 | 52 | 80% | 30% |
| 1834.946 | 53 | 45% | 5% |
| 1864.027 | 54 | 75% | 25% |
| 1875.997 | 55 | 75% | 20% |
| 1876.017 | 56 | 75% | 20% |
| 1884.000 | 57 | 65% | 20% |
| 2186.092 | 65 | 60% | 10% |
| 2296.219 | 67 | 80% | 20% |
| 2315.195 | 68 | 75% | 30% |
| 2348.245 | 70 | 95% | 30% |
| 2652.448 | 75 | 70% | 5% |
| 2670.387 | 76 | 50% | 15% |
| 2695.362 | 77 | 95% | 40% |
| 2790.425 | 78 | 55% | 15% |

Alternatively, Table 9 shows the additional peptides that may be used together with the peptide pattern of the invention and which are also capable of differentiating between arthrotic subjects and normal subjects who do not suffer from any arthrotic pathology. The difference between the data shown in Table 8 and Table 9 lies in the fact that, in Table 8, arthrotic subjects show a higher presence of said peptides, whereas the data in Table 9 show that arthrotic subjects have a lower presence of said peptides as compared to the healthy control subjects.

TABLE 9
Masses of the additional peptides, other than the peptide pattern
of the invention, useful for classifying and diagnosing patients
suffering from arthrosis (OA), and distinguishing them from
subjects who do not suffer from any arthrotic pathology. The
percentages indicate the presence of each peptide in the total
samples analysed for each group of patients.

| m/z | SEQ ID No. | OA (n = 20) | N (n = 20) |
|---|---|---|---|
| 871.502 | 12 | 0% | 30% |
| 1518.710 | 34 | 15% | 55% |
| 1810.992 | 50 | 5% | 40% |
| 1922.877 | 60 | 5% | 50% |
| 2012.011 | 62 | 40% | 80% |
| 2153.112 | 64 | 30% | 90% |
| 2602.358 | 74 | 35% | 65% |

Table 10 shows the additional peptides that may be used jointly with the peptide pattern of the invention and which are capable of differentiating between arthrotic subjects and subjects who suffer from rheumatoid arthritis. It is worth noting that, as may observed in said Table 10, subjects suffering from OA present a higher presence of the peptides with the sequences SEQ ID NO: 30, 59 and 69, as compared to those patients who suffer from RA. On the contrary, arthrotic subjects present a lower presence of the peptides with the sequences SEQ ID NO: 22, 23, 26, 31, 35, 39, 45, 46, 47, 48, 58, 61, 63, 66, 71, 72 and 73, as compared to those subjects who suffer from RA.

TABLE 10
Masses of the additional peptides, other than the peptide pattern
of the invention, useful for classifying and diagnosing patients
suffering from arthrosis (OA) and distinguishing them from
subjects who suffer from rheumatoid arthritis (RA). The percentages
indicate the presence of each peptide in the total samples
analysed for each group of patients.

| m/z | SEQ ID No. | RA (n = 20) | OA (n = 20) |
|---|---|---|---|
| 1247.652 | 22 | 50% | 25% |
| 1249.655 | 23 | 30% | 0% |
| 1299.609 | 26 | 90% | 20% |
| 1465.689 | 30 | 30% | 80% |
| 1467.852 | 31 | 90% | 45% |
| 1519.750 | 35 | 30% | 0% |
| 1612.849 | 39 | 55% | 20% |
| 1736.970 | 45 | 35% | 0% |
| 1743.882 | 46 | 45% | 10% |
| 1745.901 | 47 | 45% | 5% |
| 1771.885 | 48 | 90% | 25% |
| 1899.060 | 58 | 55% | 15% |
| 1906.873 | 59 | 5% | 80% |
| 1932.038 | 61 | 45% | 0% |
| 2020.138 | 63 | 60% | 15% |
| 2190.112 | 66 | 85% | 20% |
| 2344.169 | 69 | 5% | 80% |
| 2394.298 | 71 | 60% | 10% |
| 2409.281 | 72 | 50% | 0% |
| 2565.322 | 73 | 70% | 20% |

Table 11 shows the additional peptides that may be used together with the peptide pattern of the invention and which are capable of differentiating between arthrotic subjects and subjects who suffer from psoriatic arthritis. It is worth noting that, as may be observed in said Table 11, subjects suffering from OA present a higher presence of the peptides with the sequences SEQ ID NO: 25, 29, 30, 59 and 69, as compared to those patients who suffer from PsA. On the contrary, arthrotic subjects present a lower presence of the peptides with the sequences SEQ ID NO: 22, 26, 27, 31, 37, 39, 46, 47, 48, 49, 58, 66 and 79, as compared to those subjects who suffer from PsA.

TABLE 11
Masses of the additional peptides, other than the peptide pattern
of the invention, useful for classifying and diagnosing patients
suffering from arthrosis (OA) and distinguishing them from
subjects who suffer from psoriatic arthritis (PsA). The percentages
indicate the presence of each peptide in the total samples
analysed for each group of patients.

| m/z | SEQ ID No. | PsA (n = 20) | OA (n = 20) |
|---|---|---|---|
| 1247.652 | 22 | 55% | 25% |
| 1263.683 | 25 | 10% | 60% |
| 1299.609 | 26 | 90% | 20% |
| 1380.780 | 27 | 30% | 0% |
| 1449.800 | 29 | 25% | 75% |
| 1465.689 | 30 | 5% | 80% |
| 1467.852 | 31 | 95% | 45% |
| 1569.907 | 37 | 50% | 0% |
| 1612.849 | 39 | 75% | 20% |
| 1743.882 | 46 | 55% | 10% |
| 1745.901 | 47 | 25% | 5% |
| 1771.885 | 48 | 95% | 25% |
| 1786.909 | 49 | 65% | 20% |
| 1899.060 | 58 | 50% | 15% |
| 1906.873 | 59 | 0% | 80% |
| 2190.132 | 66 | 70% | 20% |
| 2344.169 | 69 | 0% | 80% |
| 2940.755 | 79 | 60% | 20% |

### 2.3. Principal Component Analysis (PCA).

Using the peptides selected in the peptide profile described in the invention, obtained from the serum samples from the group of arthrotic patients and the samples from the control group, a principal component analysis (PCA) was performed, which verified that the samples are separated into at least two planes under all elution conditions, such that the peptides are capable of differentiating between samples from arthrotic donors and those from the control subjects.

Prior to performing the PCA, a single data set was created for each elution percentage (4%, 7%, 10% and 14%), with the representative spectrum for each sample. The spectra of each set of data were converted into 1s and 0s vectors, where 1 means presence of a peak and 0 means absence of a peak.

The PCA analysis and the visualisation thereof were performed using the RapidMiner v5.3 programme (http://rapid-i,com/content/view/181/190/), which configures the PCA algorithm in order to reduce the dimensionality of the data sets to 3 principal components.

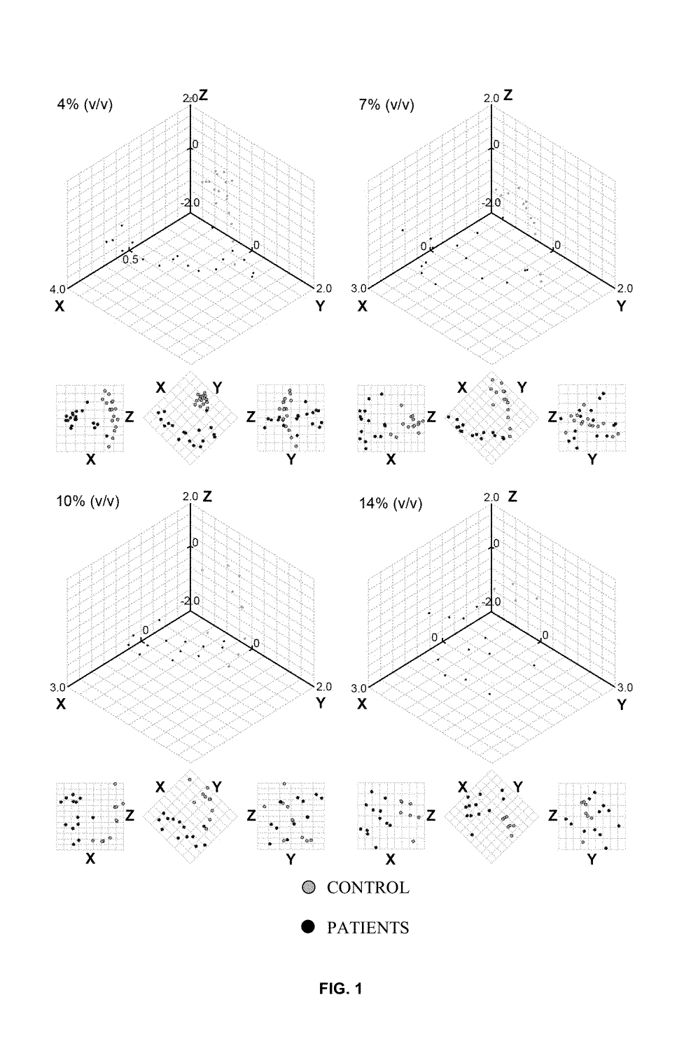

These results are shown in FIG. 1.

2.4. Testing of the Classifier with Blind Samples

The utility of the peptides that comprise the peptide pattern described in the present invention for the diagnosis of arthrosis was tested in a group of patients whose pathology was unknown (blind samples). The samples from said patients (n=79) were processed in the same manner as that described above in the materials and methods section.

Using the set of data described in section 2.1, a number of automatic classifiers were evaluated using the Weka 3.7 machine learning tool (http://www.cs.waikato.ac.nz/ml/weka/). These classifiers were trained with the different possible combinations of the data from each elution. This evaluation was performed using the cross-validation scheme known as Leave-One-Out on two different problems: i) classification of the samples under all conditions, and ii) classification of those samples that present a condition related to a pathology (RA, PsA and OA groups). As a result of this evaluation, the best classifier selected for the problem of classifying all the conditions was the LibSVM implementation of the Support Vector Machine (SVM) algorithm with a Radial Basis Function (RBF) kernel trained with the data from the 7% and 10% elution percentages, combined with an optimiser based on a grid search (GridSearch algorithm) configured to search for the optimal value of the “cost” parameter amongst the values 2⁻⁵, 2⁻³, 2⁻¹, 2¹, 2³, 2⁵, 2⁷, 2⁹, 2¹¹, 2¹³ and 2¹⁵, and the optimal value of the “gamma” parameter amongst the values 2⁻¹⁵, 2⁻¹³, 2⁻¹¹, 2⁻⁹, 2⁻⁷, 2⁻⁵, 2⁻³, 2⁻¹, 2¹ and 2³. On the other hand, the Artificial Neural Network (ANN) classifier trained with the data from the 10% percentages was selected as the best classifier for the problem of classifying the pathological conditions.

Using these classifiers trained with the set of data described in section 2.1, the set of data from the blind samples were classified. In the case of the SVM-RBF classifier for the problem of all conditions, the classifying power was only moderate, since it resulted in an accuracy ratio slightly greater than 60% and an approximate Cohen's statistic value (kappa) of 0.48. On the other hand, the ANN classifier for the problem of pathological conditions showed better results, with an accuracy ratio close to 75% and a kappa value of 0.619. The results of the classification are shown in Table 12.

TABLE 12
Automatic classifiers created using the peptide pattern described
in the present invention, obtained from the serum samples analysed
for each group of patients (n = 20 for each group of patients).

| Conds. | Clas. | Elu. | % Ac. | K | Cond. | Sen. | Sp. | n | N | CD | CDP |
|---|---|---|---|---|---|---|---|---|---|---|---|
| All | SVM- | 7% | 60.76 | 0.477 | PsA | 0.80 | 0.93 | 20 | 79 | 48 | 16 |
| | RBF- | 10% | | | RA | 1.00 | 0.92 | 19 | | | 19 |
| | OPT | | | | OA | 0.45 | 0.75 | 20 | | | 9 |
| | | | | | N | 0.20 | 0.88 | 20 | | | 4 |
| Pathologies | ANN | 10% | 74.58 | 0.619 | PsA | 0.55 | 0.90 | 20 | 59 | 44 | 11 |
| | | | | | RA | 0.89 | 0.38 | 19 | | | 17 |
| | | | | | OA | 0.80 | 0.23 | 20 | | | 16 |

Conds.: conditions, Clas.: classifier, Elu.: elution percentages, % Ac.: accuracy ratio, K: global kappa, Cond.: condition, Sen.: sensitivity by condition, Sp.: specificity by condition, n: samples per condition, N: total number of samples, CD: correct diagnosis, CDP: correct diagnosis by pathology.

1. (canceled)

2. (canceled)

3. (canceled)

4. Method for obtaining useful data for the diagnosis and classification of rheumatic diseases, which comprises:
a. obtaining an isolated biological sample from a subject, and
b. simultaneously detecting, in the isolated biological sample of (a), the expression level and/or quantity of peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11.

5. Method according to claim 4, further comprising determining, in step (b), the expression level and/or quantity of at least one of the peptides selected from the list consisting of: SEQ ID NO: 12; SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 34, SEQ ID NO: 36, SEQ ID NO: 38, SEQ ID NO: 40, SEQ ID NO: 41, SEQ ID NO: 42, SEQ ID NO: 43, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 60, SEQ ID NO: 62, SEQ ID NO: 64, SEQ ID NO: 65, SEQ ID NO: 67, SEQ ID NO: 68, SEQ ID NO: 70, SEQ ID NO: 74, SEQ ID NO: 75, SEQ ID NO: 76, SEQ ID NO: 77, SEQ ID NO: 78, SEQ ID NO: 22, SEQ ID NO: 23, SEQ ID NO: 26, SEQ ID NO: 30, SEQ ID NO: 31, SEQ ID NO: 35, SEQ ID NO: 39, SEQ ID NO: 45, SEQ ID NO: 46, SEQ ID NO: 47, SEQ ID NO: 48, SEQ ID NO: 58, SEQ ID NO: 59, SEQ ID NO: 61, SEQ ID NO: 63, SEQ ID NO: 66, SEQ ID NO: 69, SEQ ID NO: 71, SEQ ID NO: 72, SEQ ID NO: 73, SEQ ID NO: 25, SEQ ID NO: 27, SEQ ID NO: 29, SEQ ID NO: 37, SEQ ID NO: 49, SEQ ID NO: 79, or combinations thereof.

6. Method according to claim 4, wherein the rheumatic diseases are selected from any of the following: arthrosis, rheumatoid arthritis and psoriatic arthritis.

7. Method for diagnosing and classifying arthrosic diseases, comprising steps (a) and (b) according to claim 4, and further comprising:
(c) comparing the quantities obtained in step (b) for the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 34, SEQ ID NO: 36, SEQ ID NO: 38, SEQ ID NO: 40, SEQ ID NO: 41, SEQ ID NO: 42, SEQ ID NO: 43, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 60, SEQ ID NO: 62, SEQ ID NO: 64, SEQ ID NO: 65, SEQ ID NO: 67, SEQ ID NO: 68, SEQ ID NO: 70, SEQ ID NO: 74, SEQ ID NO: 75, SEQ ID NO: 76, SEQ ID NO: 77 and SEQ ID NO: 78, with a reference quantity from a sample obtained from a healthy control subject, and
(d) assigning the subjects of step (a) to the group of subjects suffering from arthrosis when they present:
(i) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, detected in (b), or
(ii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
(iii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b),
as compared to the expression level and/or the quantity of the same peptides detected in a reference sample obtained from a healthy control subject.

8. Method for diagnosing and classifying arthrosic diseases, comprising steps (a) and (b) according to claim 4, and further comprising:
(c) comparing the quantities obtained in step (b) for the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 34, SEQ ID NO: 50, SEQ ID NO: 60, SEQ ID NO: 62, SEQ ID NO: 64, SEQ ID NO: 74, SEQ ID NO: 22, SEQ ID NO: 23, SEQ ID NO: 26, SEQ ID NO: 31, SEQ ID NO: 35, SEQ ID NO: 39, SEQ ID NO: 45, SEQ ID NO: 46, SEQ ID NO: 47, SEQ ID NO: 48, SEQ ID NO: 58, SEQ ID NO: 61, SEQ ID NO: 63, SEQ ID NO: 66, SEQ ID NO: 71, SEQ ID NO: 72, SEQ ID NO: 73, SEQ ID NO: 30, SEQ ID NO: 59, SEQ ID NO: 69, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, with a reference quantity from a sample obtained from a subject suffering from arthrosis, and
(d) assigning the subjects of step (a) to the group of subjects suffering from rheumatoid arthritis when they present:
(i) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 22, SEQ ID NO: 23, SEQ ID NO: 26, SEQ ID NO: 31, SEQ ID NO: 35, SEQ ID NO: 39, SEQ ID NO: 45, SEQ ID NO: 46, SEQ ID NO: 47, SEQ ID NO: 48, SEQ ID NO: 58, SEQ ID NO: 61, SEQ ID NO: 63, SEQ ID NO: 66, SEQ ID NO: 71, SEQ ID NO: 72 and SEQ ID NO: 73, detected in (b); or
(ii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11, and a significantly lower expression level and/or quantity of the peptides with the sequences SEQ ID NO: 30, SEQ ID NO: 59 and SEQ ID NO: 69, detected in (b); or
(iii) a significantly higher expression level and/or quantity of the peptides of step (i), and the peptides with the sequences SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, detected in (b); or
(iv) a significantly higher expression level and/or quantity of the peptides of step (i), and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
(v) a significantly higher expression level and/or quantity of the peptides of step (iii), and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
(vi) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, and a significantly lower expression level and/or quantity of the peptides with the sequences SEQ ID NO: 30, SEQ ID NO: 59 and SEQ ID NO: 69, detected in (b); or
(vii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, SEQ ID NO: 30, SEQ ID NO: 59 and SEQ ID NO: 69, detected in (b); or
(viii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, SEQ ID NO: 30, SEQ ID NO: 59 and SEQ ID NO: 69,
as compared to the expression level and/or the quantity of the same peptides detected in a reference sample obtained from a subject suffering from arthrosis.

9. Method for diagnosing and classifying arthrosic diseases, comprising steps (a) and (b) according to claim 4, and further comprising:
(c) comparing the quantities obtained in step (b) for the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 22, SEQ ID NO: 26, SEQ ID NO: 27, SEQ ID NO: 31, SEQ ID NO: 37, SEQ ID NO: 39, SEQ ID NO: 46, SEQ ID NO: 47, SEQ ID NO: 48, SEQ ID NO: 49, SEQ ID NO: 58, SEQ ID NO: 66, SEQ ID NO: 79, SEQ ID NO: 25, SEQ ID NO: 29, SEQ ID NO: 30, SEQ ID NO: 59, SEQ ID NO: 69, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77, SEQ ID NO: 78, SEQ ID NO: 12, SEQ ID NO: 34, SEQ ID NO: 50, SEQ ID NO: 60, SEQ ID NO: 62, SEQ ID NO: 64 and SEQ ID NO: 74, with a reference quantity from a sample obtained from an subject suffering from arthrosis, and
(d) assigning the subjects of step (a) to the group of subjects suffering from psoriatic arthritis when they present:
(i) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 22, SEQ ID NO: 26, SEQ ID NO: 27, SEQ ID NO: 31, SEQ ID NO: 37, SEQ ID NO: 39, SEQ ID NO: 46, SEQ ID NO: 47, SEQ ID NO: 48, SEQ ID NO: 49, SEQ ID NO: 58, SEQ ID NO: 66 and SEQ ID NO: 79, detected in (b); or
(ii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11, and a significantly lower expression level and/or quantity of the peptides with the sequences SEQ ID NO: 25, SEQ ID NO: 29, SEQ ID NO: 30, SEQ ID NO: 59 and SEQ ID NO: 69, detected in (b); or
(iii) a significantly higher expression level and/or quantity of the peptides of step (i), and the peptides with the sequences SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, detected in (b); or
(iv) a significantly higher expression level and/or quantity of the peptides of step (i), and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
(v) a significantly higher expression level and/or quantity of the peptides of step (iii), and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, detected in (b); or
(vi) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, and a significantly lower expression level and/or quantity of the peptides with the sequences SEQ ID NO: 25, SEQ ID NO: 29, SEQ ID NO: 30, SEQ ID NO: 59 and SEQ ID NO: 69, detected in (b); or
(vii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, SEQ ID NO: 25, SEQ ID NO: 29, SEQ ID NO: 30, SEQ ID NO: 59 and SEQ ID NO: 69, detected in (b); or
(viii) a significantly higher expression level and/or quantity of the peptides with the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 36, SEQ ID NO: 38, SEQ ID NO: 40, SEQ ID NO: 41, SEQ ID NO: 42, SEQ ID NO: 43, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 65, SEQ ID NO: 67, SEQ ID NO: 68, SEQ ID NO: 70, SEQ ID NO: 75, SEQ ID NO: 76, SEQ ID NO: 77 and SEQ ID NO: 78, and a significantly lower expression level and/or quantity of the peptides with the sequences SEQ ID NO: 12, SEQ ID NO: 34, SEQ ID NO: 50, SEQ ID NO: 60, SEQ ID NO: 62, SEQ ID NO: 64 and SEQ ID NO: 74, SEQ ID NO: 25, SEQ ID NO: 29, SEQ ID NO: 30, SEQ ID NO: 59 and SEQ ID NO: 69, detected in (b);
as compared to the expression level and/or the quantity of the same peptides detected in a reference sample from a group of subjects suffering from arthrosis.

10. Method according to claim 7, wherein steps (b) and/or (c) are totally or partially automated.

11. Method according to claim 4, wherein the sample is a biological fluid.

12. Method according to claim 11, wherein the biological fluid is selected from the group consisting of blood, serum, plasma, synovial fluid and urine.

13. Method according to claim 4, wherein the detection of the expression levels and/or quantity of the peptides is performed by means of mass spectrometry, ELISA, array, microarray, flow cytometry, mass spectrometry, gas chromatography-mass spectrometry or liquid chromatography-mass spectrometry.

14. Kit or device for diagnosing and classifying rheumatic diseases, comprising the necessary molecular tools to simultaneously detect the expression level and/or quantity of the peptides with the sequences SEQ ID NO: 1 to 11.

15. Kit according to claim 14, further comprising the necessary molecular tools to detect the expression level and/or quantity of at least one of the peptides with the sequences: SEQ ID NO: 12; SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 24, SEQ ID NO: 28, SEQ ID NO: 32, SEQ ID NO: 33, SEQ ID NO: 34, SEQ ID NO: 36, SEQ ID NO: 38, SEQ ID NO: 40, SEQ ID NO: 41, SEQ ID NO: 42, SEQ ID NO: 43, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 60, SEQ ID NO: 61, SEQ ID NO: 62, SEQ ID NO: 64, SEQ ID NO: 65, SEQ ID NO: 66, SEQ ID NO: 67, SEQ ID NO: 68, SEQ ID NO: 70, SEQ ID NO: 74, SEQ ID NO: 75, SEQ ID NO: 76, SEQ ID NO: 77, SEQ ID NO: 78, SEQ ID NO: 22, SEQ ID NO: 26, SEQ ID NO: 30, SEQ ID NO: 31, SEQ ID NO: 35, SEQ ID NO: 39, SEQ ID NO: 45, SEQ ID NO: 46, SEQ ID NO: 47, SEQ ID NO: 48, SEQ ID NO: 58, SEQ ID NO: 59, SEQ ID NO: 61, SEQ ID NO: 63, SEQ ID NO: 66, SEQ ID NO: 69, SEQ ID NO: 71, SEQ ID NO: 72, SEQ ID NO: 73, SEQ ID NO: 25, SEQ ID NO: 27, SEQ ID NO: 29, SEQ ID NO: 37, SEQ ID NO: 49, SEQ ID NO: 69, SEQ ID NO: 79, or combinations thereof.

16. Kit according to claim 14, wherein the molecular tools capable of detecting the expression level and/or quantity of the peptides are selected from any of the following: antibodies or fragments thereof, and oligonucleotides capable of hybridising to the nucleotide sequences that encode the peptides.

17. Kit according to claim 15, wherein the rheumatic diseases are selected from any of the following: arthrosis, rheumatoid arthritis and psoriatic arthritis.

18-21. (canceled)

22. Method according to claim 8, wherein steps (b) and/or (c) are totally or partially automated.

23. Method according to claim 9, wherein steps (b) and/or (c) are totally or partially automated.

24. Method according to claim 4, wherein step (b) is totally or partially automated.
